(19) United States
(12) Patent Application Publication
(10) Pub. No.: US 2007/0099882 A1
(43) Pub. Date: May 3, 2007

(54) Methods and compositions for prolonged alleviation of articular joint pain

(76) Inventors: Harry C. Gurney, Evergreen, CO (US); Janice Gurney, Evergreen, CO (US)

Correspondence Address: PATTON BOGGS, L.L.P., 2001 ROSS AVENUE, SUITE 3000, DALLAS, TX, 75201, US

(21) Appl. No.: 11/260,726

(22) Filed: Oct. 27, 2005

(51) Int. Cl. A61K 31/573; A61K 31/24

(52) U.S. Cl. 514/171; 514/534

(57) ABSTRACT

The methods and compositions for prolonged alleviation of articular, synovia, and connective tissue joint pain include treatment solutions that are prepared and administered into the joint space or cavity of a painful (arthritic) or connective tissue target of the patient. The compositions include Procaine and at least two corticosteroids for providing prolonged alleviation of articular joint pain. A painful articular joint of a patient is positioned to facilitate the administration of the treatment solution into the joint or synovia space. The skin area over the joint space, is disinfected prior to the administration of the treatment solution. The administration of the treatment solution can be by injection with a syringe needle. Several different methods are provided for prolonged alleviation of pain of several different articular joints. The methods and compositions for prolonged alleviation of articular joint pain also can include kits for the treatment of such joint pain.

### FIELD OF THE INVENTION

This invention relates to methods and compositions for prolonged alleviation of chronic articular joint pain due to arthritis or other chronic inflammatory processes involving joints and joint tissue structures using an injectable mixture of an anesthetic and selected corticosteroids.

### PROBLEM

There are many medications, remedies, chiropractic, and traditional Chinese medical manipulative interventions that are used to treat the pain associated with articular joint abnormalities, chronic inflammation, and arthritis. The available interrogative methods generally available for localizing the specific foci of pain and medical/surgical treatments may fail to lead to relatively long term pain relief.

There exists difficulties that are often encountered in the identification of the specific foci and pain triggers, preoperative and postoperative, involving joint structures of the body, including the spine, which are commonly examined by palpation, manipulation, X-ray, cat scan, and MRI whereby lesions of variable duration may be identified but are not necessarily the area of pain. Such abnormal findings may or may not accurately localize the specific foci of pain production nor the bio-molecular cause of pain as some tissue accretions may exist for indeterminate periods of time without causing pain.

In addition, among the common protocols for treating articular joint pain is through surgical and specific medical interventions that subject patients to unfavorable risks.

The traditional use of corticosteroid joint injections alone or in combination with local anesthetics, such as Lidocaine and other local anesthetics, do not consistently confer long term pain relief. The doses required to obtain pain relief of a temporary duration are limited by the potential for iatrogenic complications involving adrenal gland function (Cushing's disease). Additionally, it is observed that the benefits in relieving joint pain using standard Lidocaine or Marcaine combinations with high dosages of single joint compatible corticosteroids are variable in response and commonly of short duration. Others have attempted to alleviate articular joint pain by injecting a mixture containing a single corticosteroid with an anesthetic with limited results.

Information relevant to attempts to address these problems can be found in the U.S. Patent Application Nos. 20040047807 filed 11 Aug. 2003 by Meyer; 20040152713 filed 21 Jan. 2004 by Petrie; 20040022847 filed 29 Jul. 2003 by Leneau; and 20030232784 filed 14 Jun. 2002 by Benedict.

Therefore, there is a need for treatment methods and compositions for alleviating articular joint pain with prolonged effects that does not cause adrenal gland complications and that does not require surgery.

### SOLUTION

The above described problems are solved and a technical advance achieved by the methods and compositions for prolonged alleviation of articular joint pain that provide a method for localizing specific anatomical sites of pain while concurrently providing an extended period of relief from pain.

The locations of pain appropriate for this method include joint tissues, calcified connective tendons, ligaments associated joint structures, nerve impingements associated with local inflammation, chronic cellular tissue restrictions which interfere with joint mobility, range of motion, and arthritic aggregates.

The method for prolonged alleviation of articular joint pain specifically identifies the trigger location of pain and in this regard becomes a diagnostic tool. When the invention is applied by local injections the pain is relieved for substantially prolonged periods of time, often eliminating the necessity of using systemic drugs or other interventions. In addition, this method is designed to address the problem of chronic pain in cases where surgical and specific medical interventions have not been successful and cases where the patient is put to unfavorable risk.

The method is based upon the discovery that very small doses of the local anesthetic, such as 4% Procaine, in combination with joint compatible corticosteroids, such as Depomedrol and Betamethasone, administered by intra articular injection provides relief from acute joint pain or associated tissue structures and the duration of relief from pain is prolonged for several years.

The mechanism of action of Procaine when used in combination with appropriate joint compatible corticosteroids is clearly different clinically from that of other local anesthetics, which provide temporary relief at best. The use of the method for prolonged alleviation of articular joint pain in cases of severe capsular or tissue fibrosis and osteogenic proliferations is most effective when injected into the joint space or areas of inflammatory impingement.

The methods and compositions for prolonged alleviation of articular joint pain alleviate chronic and acute joint pain for a period of years when injected into the intro articular joint space and other areas of chronic pain.

### SUMMARY

The invention provides methods and compositions for alleviating articular and connective tissue pain in a joint of a patient, the method comprising preparing a treatment solution containing at least one anesthetic and at least two joint, connective tissue, or synovia compatible corticosteroids, and administering said treatment solution through an area of skin the target area. Preferably, the method further comprises disinfecting the area of skin of the patient prior to the administering of the treatment solution. Preferably, the at least one anesthetic is Procaine. Preferably, the at least two joint, connective tissue, or synovia compatible corticosteroid is selected from the group consisting of Betamethasone, Depomedrol, and Cortisone acetate. Preferably, the at least two joint, connective tissue, or synovia compatible corticosteroids are Betamethasone and Depomedrol. Preferably, the at least one anesthetic is present in the treatment solution in the range of from about 10 to about 70 percent by volume, based on the total volume of the treatment solution. Preferably, the at least one anesthetic is present in the treatment solution in the range of from about 30 to about 50 percent by volume, based on the total volume of the treatment solution. Preferably, the at least one anesthetic is 40 percent by volume in the treatment solution, based on the total volume of the treatment solution. Preferably, the at least two corticosteroid is each present in the treatment solution in the range of from about 10 to about 50 percent by volume, based on the total volume of the treatment solution. Preferably, the at least two corticosteroid is each present in the treatment solution in the range of from about 20 to about 40 percent by volume, based on the total volume of the treatment solution. Preferably, the at least two corticosteroid is each present in the treatment solution at 30 percent by volume, based on the total volume of the treatment solution. Preferably, the administering comprises injecting the treatment solution into the joint of the patient. Preferably, the injecting is performed by a needle and syringe. Preferably, the needle is between ¾ and 1½ inches in length. Preferably, the needle is between 22 and 25 gauge in diameter. Preferably, the joint is selected from the group consisting of scapular humeral joint, elbow joint, carpal joint, stifle joint, coxofemoral joint, temporomandibular joint, and hock joint. Preferably, the administering further comprises administering a first treatment solution into the joint cavity of the joint and then administering a second treatment solution into the joint cavity of the joint approximately thirty days later.

### BRIEF DESCRIPTION OF THE DRAWINGS

FIG. 1 illustrates an infusion of a scapular humeral joint and bicipital tendon slightly flexed depicting the approach of a syringe needle from an anterior lateral direction to the joint capsule with the joint slightly flexed;

FIG. 2 illustrates an infusion of an elbow joint flexed at 90 degrees depicting the approach of a syringe needle from the posterior lateral direction; and

FIG. 3 illustrates an infusion of a carpal joint slightly flexed depicting an anterior approach of a syringe needle to the medial or lateral joint compartments;

FIG. 4 illustrates an infusion of a stifle joint depicting an anterior approach of a syringe needle to the patellar ligament;

FIG. 5 illustrates an infusion of a coxofemoral joint depicting an anterior approach of a syringe needle to the greater trochanter;

FIG. 6 illustrates an infusion of a hock joint depicting a posterior approach of a syringe needle to the tibial/tarsal joint;

FIG. 7 illustrates several different infusions of a leg bone of a horse depicting posterior and anterior approaches of a syringe needle to the joints;

FIG. 8 illustrates additional injection infusions of a leg bone of a horse depicting posterior and anterior approaches of a syringe needle to the joints;

FIG. 9 illustrates two different infusions of a shoulder joint of a human depicting an anterior and a posterior approach of a syringe needle to the joint;

FIG. 10 illustrates three different infusions of a hip joint of a human depicting dorsal and anterior approaches of a syringe needle to the joint; and

FIG. 11 illustrates in flow diagram form a method for prolonged alleviation of articular joint pain.

### DETAILED DESCRIPTION

The terms “treat”, “treatment,” and “treating” include preventative (e.g., prophylactic) and palliative treatment or the act of providing preventative or palliative treatment. In the present method for prolonged alleviation of chronic articular joint pain, these terms include the amelioration of joint pain, which means the methods of the present invention are effective in reducing the intensity of the joint pain. The term “pain” is used in the general sense and is meant to encompass pain levels from the merely uncomfortable to the virtually unbearable. The term “joint pain” is used to identify pain in the joint areas of the afflicted patient, such as, but not limited to, pain in the finger, toe, wrist, elbow, shoulder, hip, knee, or ankle joints. The term “patient” means animals, particularly mammals and including humans.

The term “joint cavity,” joint space,” and “joint capsule” mean a hollow place or depression in the place of union or junction between two or more bones of the skeleton, including the space of a synovial joint, enclosed by the synovial membrane and articular cartilages.

The term “anesthetics” means generally compounds that are characterized by or produce anesthesia, particularly drugs and agents used to abolish the sensation of pain. This includes agents whose anesthetic action is limited to an area of the body determined by the site of its application or injection. The term “corticosteroid” means any of the steroids elaborated by the adrenal cortex, excluding the sex hormones of adrenal origin, in response to the release of corticotropin by the pituitary gland and the synthetic equivalents of these steroids. The term “injection” means the act of forcing liquid into a part, as into the subcutaneous tissues, of the body, particularly with a syringe needle.

The present methods and compositions for prolonged alleviation of chronic articular joint pain due is both diagnostic and therapeutic. Regarding the former, many patients presented with joint pain, very often are found to have locations clearly painful and other locations which manifest questionable sensitivity upon palpation and manipulation. It has been discovered that if the most painful areas located are injected with the treatment composition, a dramatic improvement is noted if that site is primary. On the other hand, if a secondary location is also causing pain, then the symptoms of lameness may shift from anterior to posterior or from side to side away from the originally injected locations. Also, in cases where there is a neurological component causing debilitation, nullifying the joint symptoms will not be successful. Based upon these observations, it is important to either perform anesthesia of the specific joints in sequence or utilize the invention sequentially to clearly establish efficacy and accuracy of diagnosis. The present methods and compositions for prolonged alleviation of chronic articular joint pain is therapeutic for patient's with joint pain. For example, when injected with the treatment composition, the patient will manifest clear relief from pain for ten to fourteen days, which may be followed by the return of some level of pain until preferably, the second injection is administered preferably thirty days after the first injection.

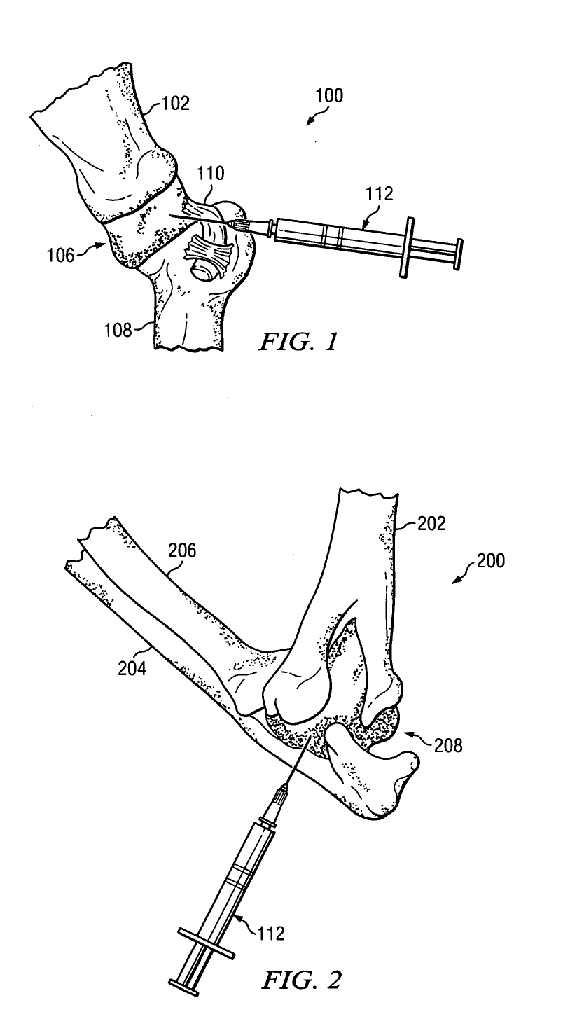

FIG. 1 illustrates an embodiment of an infusion of a scapular humeral (shoulder) joint 100 and bicipital tendon 110 that is slightly flexed showing the approach of a syringe needle 112 from an anterior lateral direction to the joint capsule 106. The scapular humeral joint 100 includes scapula 102 and the humerus 108.

FIG. 2 illustrates an embodiment of an infusion of an elbow joint 200 flexed at approximately 90 degrees showing the approach of the syringe needle 112 from the posterior lateral direction to the joint capsule 208. The elbow joint 200 includes humerus 202, ulna 204, and radius 206.

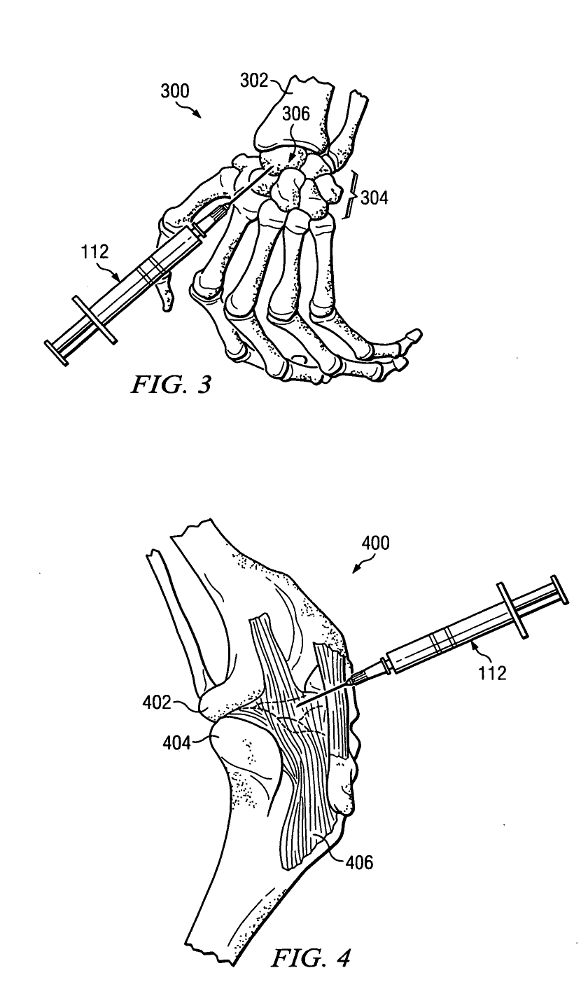

FIG. 3 illustrates an embodiment an infusion of a carpal joint (wrist) 300 slightly flexed depicting an anterior approach of the syringe needle 112 to the medial or lateral joint compartments 306. The carpal joint 300 includes the radius 302 and carpals 304.

FIG. 4 illustrates an embodiment of an infusion of a stifle joint (knee) 400 depicting an anterior approach of the syringe needle 112 to the patellar ligament 406. The stifle joint 400 includes the lateral condyle of femur 402 and the head of the fibula 404.

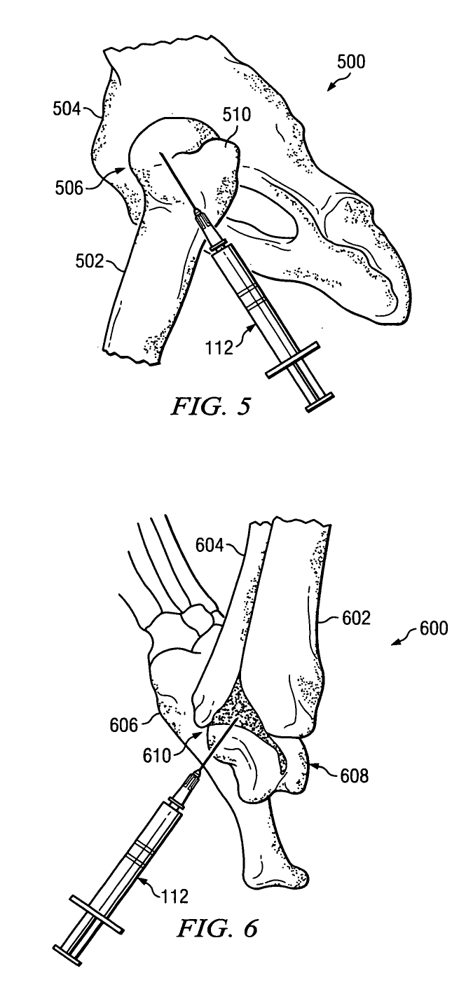

FIG. 5 illustrates an embodiment of an infusion of a coxofemoral joint (hip) 500 depicting an anterior approach of the syringe needle 112 to the greater trochanter 510. The coxofemoral joint 500 includes the head 506 of the femur 502, the pelvis 504, and the greater trochanter 510 of the femur 502.

FIG. 6 illustrates an embodiment an infusion of a hock joint (ankle) 600 depicting an posterior approach of the syringe needle 112 to the tibial/tarsal joint 608. The tibial/tarsal joint 608 includes the tibia 602, fibula 604, tibial/tarsal bone 606, and the lateral joint articulation 610.

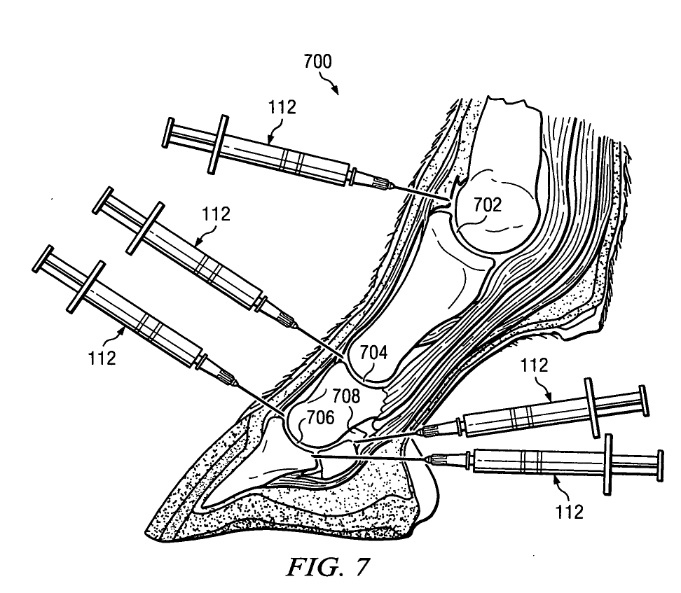

FIG. 7 illustrates an embodiment of an infusion of a leg foot (700) of a horse depicting posterior and anterior approaches of the syringe needle 112 to several different joints of the leg foot. Though several injection sites of the syringe needle 112 are shown, in one aspect of FIG. 7 each is separate and independent from the others may be administered separately and independently from the others. In another aspect, two or more of the injection sites of the syringe needle 112 may be accessed concurrently or sequentially to administer an effective treatment. The leg foot 700 includes a fetlock joint 702 and in one aspect of the present invention an anterior approach of the syringe needle 112 is shown to the fetlock joint as depicted in FIG. 7. Other anterior approaches of the syringe needle 112 are depicted, such as to the pastern joint 704 and the 2^{nd}-3^{rd} phalanx joint 706. FIG. 7 depicts two posterior injection approaches of the syringe needle 112 to the leg foot, notably two different injection sites to the navicular (coffin) joint 708.

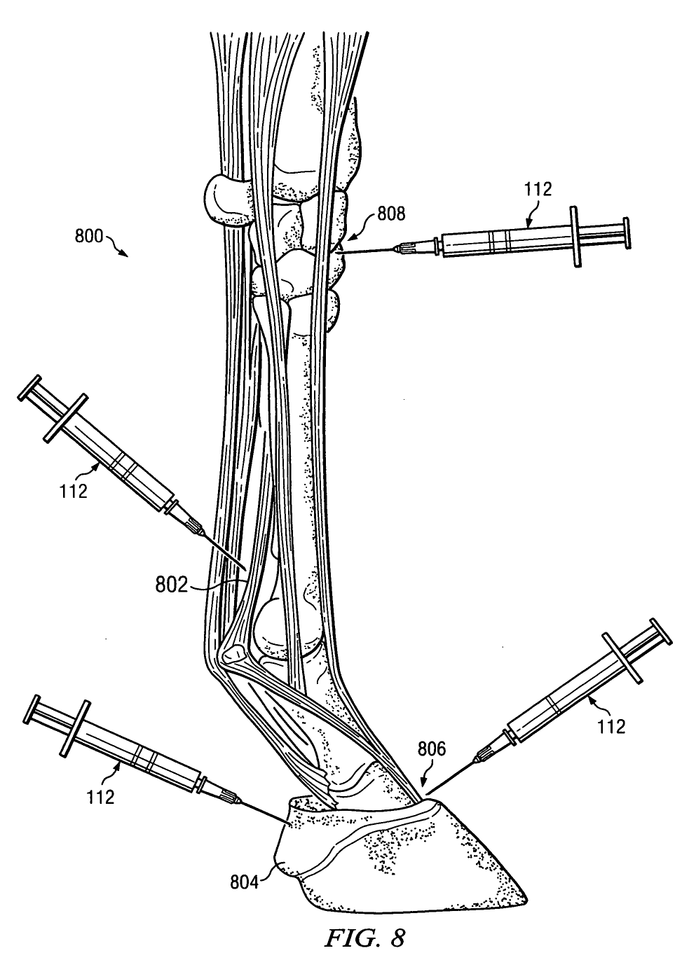

FIG. 8 illustrates an additional embodiment of of infusions of a leg bone 800 of a horse depicting posterior and anterior approaches of the syringe needle 112 to several additional different joints of the leg foot. Like FIG. 7, these approaches of the syringe needle 112 may be accessed separately and independently from each other or two or more may be accessed concurrently or sequentially. The leg foot 800 includes a bowed tension and sheath 802 and in one aspect of the present invention a posterior approach of the syringe needle 112 is shown to the bowed tension and sheath 802. Additionally, the the leg bone 800 includes a lateral-medial cartilage 804 and in one aspect can be accessed from a posterior approach with the syringe needle 112 as depicted in FIG. 8. Also, the leg foot 800 includes an arthritis 2^{nd}-3^{rd} phalanges 806 that can be accessed from a posterior approach with the needle syringe 112. Further, the leg foot 800 includes a carpal joint 808 and in one aspect of the present invention an anterior approach of the syringe needle 112 is shown to the joint.

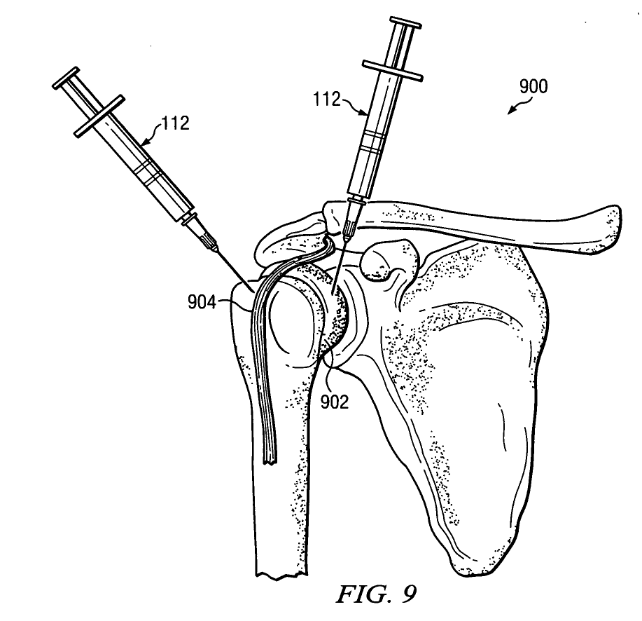

FIG. 9 illustrates an embodiment of infusions of a shoulder joint 900 of a human depicting an anterior and a posterior approach of the syringe needle 112 to the joint. The shoulder joint 900 includes a scapular-humeral joint 902 and in one aspect of the present invention an anterior approach of the syringe needle 112 is shown to the joint. Additionally, the shoulder joint 900 includes a bicipital tendon 904 and in one aspect of the present invention an anterior approach of the syringe needle 112 is shown to the tendon. These infusions may be applied individually and separately or together either concurrently or sequentially.

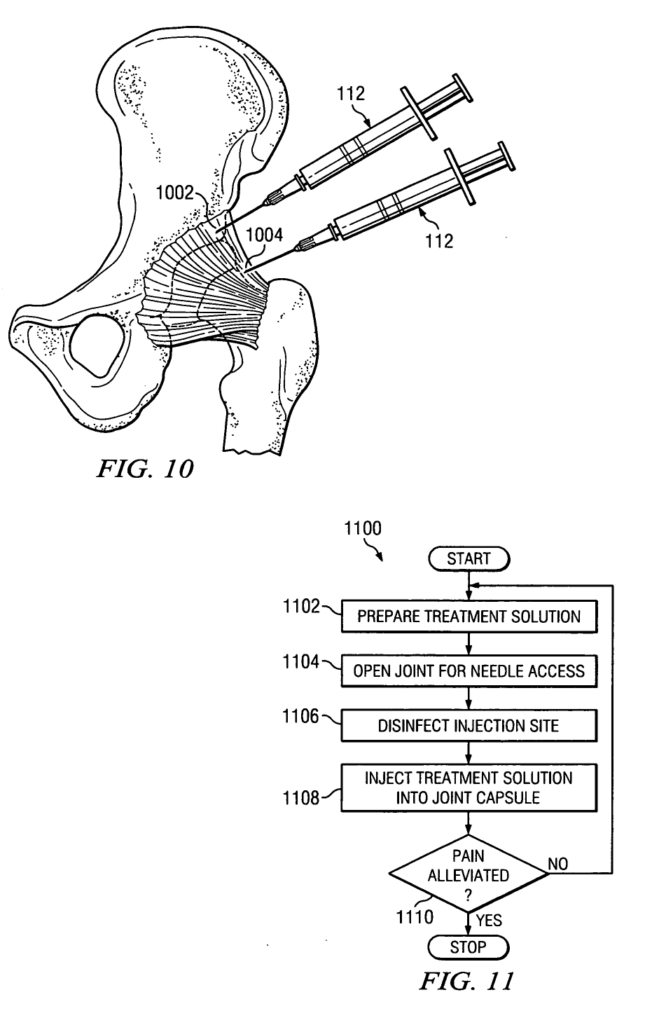

FIG. 10 illustrates an embodiment of infusions of a hip joint 1000 of a human depicting a dorsal and a anterior approach of the syringe needle 112 to the joint. In one aspect of the present invention, a posterior approach 1002 of the syringe needle 112 is shown to the joint. Additionally, another aspect of the present invention includes an anterior approach 1004 of the syringe needle 112 is shown to the joint. These infusions may be applied individually and separately or together either concurrently or sequentially,

The joints shown in FIGS. 1-10 are shown with the surrounding and covering soft tissue (skin and muscle) removed for illustration purposes. In addition to the aforementioned aspects and embodiments of the present invention, the present invention further includes methods for prolonged treating chronic articular joint pain. The present method for prolonged alleviation of articular joint pain includes administering specific solutions into the intro articular joint space and other areas of chronic pain of a patient. The method provides a treatment for localizing specific anatomical sites of pain while concurrently providing an extended period of relief from pain. The treatment solution includes a mixture of an anesthetic for pain relief in combination with at least one joint compatible corticosteroid for prolonged alleviation of articular joint pain. A solution is chosen and mixed according to the patient's locality of joint pain.

FIG. 11 illustrates one embodiment of a method 1100 for prolonged treating of chronic articular joint pain. In step 1102, a treatment solution is prepared according to the compositions herein disclosed. The solution of the present method for prolonged alleviation of articular joint pain is injected into the painful intra articular joint or synovial spaces in a volume of approximately 1.0 cubic centimeter (“cc”) in large joints, such as the carpus and ankle or larger joints, using a ¾ inch to 1½ inch or longer 22 gauge needle with preferably a 1 ml syringe needle.

The solutions used in this method, compositions and kits, are preferably administered locally, preferably by an instrument of injecting liquids into a joint cavity according to conventional methods. In one embodiment of the present method, the solution is directly injected via a syringe needle into the joint space of the painful (arthritic) or connective tissue target of the patient. The size of the syringe needle is determined by the health professional administering the solution and the amount of solution to be injected into the painful (arthritic) or connective tissue target of the patient. Preferably, the syringe is capable of containing at least 0.1 cc of liquid and administering the liquid subcutaneously into the joint space of the patient via a syringe needle.

The typical volumes of solution injected into the painful (arthritic) or connective tissue target of the patient ranges from 0.1 cc to 2.5 cc of solution per administration, depending on the size of the joint being treated. Preferably, the solution volume is ranges between 0.5 cc to 1.5 cc of solution and most preferably, the volume is 1.0 cc of solution.

The anesthetic component of the solution dosage typically comprises at least one anesthetic that preferably has a volume that is between 20% to 60% of the total solution volume of each solution dosage. More preferably, the anesthetic component is approximately 40% of the total solution volume of each solution dosage. The corticosteroid component of the solution dosage typically comprises at least two corticosteroids, each corticosteroid component preferably has a volume that is between 10% to 50% of the total solution volume of each solution dosage. More preferably, each corticosteroid component is approximately 30% of the total solution volume for each solution dosage.

The anesthetic component of the solution of the present method is selected based on the desired duration of pain relief provided to the painful (arthritic) or connective tissue target of the patient. Exemplary anesthetic compounds include procaine hydrochloride (“Procaine”). In one embodiment of the present method, Procaine is used and has the formula C₆H₄NH₂COOCH₂CH₂N(C₂H₅)₂.HCl. Preferably, the Procaine is prepared in a 4% solution and then the desired volume of this 4% solution is then mixed into the solution with the corticosteroids portion.

The joint, connective tissue, or synovia compatible corticosteroids components of the present method are selected from the group consisting of Betamethasone, Methylprednisolone acetate (“Depomedrol”), Cortisone acetate, Dexamethasone, Hydrocortisone, Methylprednisolone, Prednisolone, Prednisolone sodium phosphate, and Prednisone. In one embodiment of the present method corticosteroids component includes a mixture of Dexamethasone and Depomedrol. In one embodiment of the method the anesthetic is buffered.

The needle of the syringe needle of the present method is selected based on the size of the joint space of the painful (arthritic) or connective tissue target of the patient. In one embodiment of the present method the needle is a hypodermic needle, preferably an arthroscopic needle, but may be other types of needles depending on the application including non-arthroscopic needles. Preferably, the present method provides for needle gauges in the range of between 22 and 25 gauge and from ¾ inch to 1½ inches in length.

In step 1104, the joint is opened for syringe needle access to a joint or tendon sheath interior. This is done by manipulating the bones that define the joint such that access with a syringe needle to the joint or tendon sheath interior can be achieved.

In step 1106, the injection site on the skin is aseptically prepared in a routine fashion to maintain sterility. This is done by cleaning and preparing the site of injection using methods, techniques, and preparations commonly used in the medical arts. Some exemplary topical solutions for disinfection of the site of the injection includes alcohol and betadyne scrub.

In step 1108, the treatment solution is injected into the joint. Preferably, this is done by inserting the syringe needle through the treated topical skin area and then slowly guiding the needle of the syringe needle into the synovia capsule of the target joint. Depending upon the joint or target space capacity, preferably 1.0 ml or slightly less of the present treatment composition is introduced slowly into the joint capsule.

In step 1110, preferably, a second injection is administered 30 days after the first injection. The present method for prolonged alleviation of articular joint pain provides for administering a first injection containing a solution as described herein to the painful (arthritic) or connective tissue target of the patient to provide for a limited duration of pain alleviation. The first injection of the Procaine combination results in an initial pain reduction or elimination for a period of 10-14 days, or longer, after which discomfort or pain may return but at generally a reduced intensity. The present method further provides for administering a second injection containing the same solution in the same site as described herein to the affected joint to provide for significantly longer duration pain alleviation, for example up to 2-4 years, or longer. Experience with this procedure showed that the thirty day interval between injections is very important. Preferably the second injection is administered no sooner than 30 days from the first injection, though shorter periods of time between the two injections does provide substantially improved pain alleviation over the prior art. Additional injections into the painful (arthritic) or connective tissue target of the patient may be necessary due to the condition of the painful (arthritic) or connective tissue target of the patient.

The solution can be mixed in a separate container prior to be loaded into a syringe or they can be mixed in the syringe directly prior to administration.

By way of non-limiting examples, the following treatments are provided to further describe the present method for prolonged treating chronic articular joint pain.

### EXAMPLE 1

### Scapular Humeral Joint

A solution containing 0.4 cc of buffered Procaine combined with 0.3 cc of Depomedrol and 0.3 cc of Betamethasone was prepared and loaded into a syringe having a ¾ inch 22 gauge needle. The skin over the injection site was prepared by disinfection with an appropriate topical solution or scrub. The scapular humeral joint arthritis (shoulder) and bicipital tendon (calcified or uncalcified) was slightly flexed and the syringe needle was directed towards the joint from an anterior lateral direction to the joint space. The introduction of the solution was administered slowly into the joint space or synovial space of the joint. This comprised the first injection of the method for prolonged treating chronic articular joint pain. A second injection was prepared and administered similarly to the first injection to the joint 30 days later.

### EXAMPLE 2

### Elbow Joint

A solution containing 0.4 cc of buffered Procaine combined with 0.3 cc of Depomedrol and 0.3 cc of Betamethasone was prepared and loaded into a syringe having a ¾ inch 22 gauge needle. The skin over the injection site was prepared by disinfection with an appropriate topical solution or scrub. The elbow joint was slightly flexed approximately ninety degrees so that the joint space could be accessed either from the posterior lateral or lateral direction and the syringe needle was directed towards the joint from the posterior lateral direction to the joint space. The introduction of the solution was administered slowly into the joint space or synovial space of the joint. This comprised the first injection of the method for prolonged treating chronic articular joint pain. A second injection was prepared and administered similarly to the first injection to the joint 30 days later. In very small joints a 25 gauge needle may be substituted.

### EXAMPLE 3

### Carpal Joint

A solution containing 0.4 cc of buffered Procaine combined with 0.3 cc of Depomedrol and 0.3 cc of Betamethasone was prepared and loaded into a syringe having a ¾ inch 22 gauge needle. The skin over the injection site was prepared by disinfection with an appropriate topical solution or scrub. The carpal joint was slightly flexed so that the joint space to facilitate an anterior approach of the needle to the medial or lateral joint compartment. The introduction of the solution was administered slowly into the joint space or synovial space of the joint. This comprised the first injection of the method for prolonged treating chronic articular joint pain. A second injection was prepared and administered similarly to the first injection to the joint 30 days later. In very small joints a reduced volume of the solution may be preferred.

### EXAMPLE 4

### Stifle (Knee) Joint

A solution containing 0.4 cc of buffered Procaine combined with 0.3 cc of Depomedrol and 0.3 cc of Betamethasone was prepared and loaded into a syringe having a ¾ inch 22 gauge needle. The skin over the injection site was prepared by disinfection with an appropriate topical solution or scrub. The stifle joint was positioned to facilitate an anterior lateral or medial approach of the needle to either side of the patellar ligament. The introduction of the solution was administered slowly into the joint space or synovial space of the joint. This comprised the first injection of the method for prolonged treating chronic articular joint pain. A second injection was prepared and administered similarly to the first injection to the joint 30 days later. This joint is commonly involved in post operative and chronically destabilized joints where the cranial cruciate ligament has been ruptured, with or without damage to the medial meniscus.

### EXAMPLE 5

### Coxofemoral (Hip) Joint

A solution containing 0.4 cc of buffered Procaine combined with 0.3 cc of Depomedrol and 0.3 cc of Betamethasone was prepared and loaded into a syringe having a ¾ inch 22 gauge needle. The skin over the injection site was prepared by disinfection with an appropriate topical solution or scrub. The coxofemoral joint was slightly positioned so that the anterior edge of the greater trochanter is used as a guide point. The needle is introduced just anterior to the greater trochanter and then penetrated directly into the joint space, using the neck of the femoral head to move into the joint space. The introduction of the solution was administered slowly into the joint space or synovial space of the joint. This comprised the first injection of the method for prolonged treating chronic articular joint pain. A second injection was prepared and administered similarly to the first injection to the joint 30 days later. In this joint it is common to locate a markedly thickened joint capsular area through which the needle is moved. In very small joints a 25 gauge needle may be substituted.

### EXAMPLE 6

### Hock (Ankle) Joint

A solution containing 0.4 cc of buffered Procaine combined with 0.3 cc of Depomedrol and 0.3 cc of Betamethasone was prepared and loaded into a syringe having a ¾ inch 22 gauge needle. The skin over the injection site was prepared by disinfection with an appropriate topical solution or scrub. The hock joint (ankle) was positioned so that the joint space of the tibial or tarsal joint to facilitate a posterior approach of the needle to the medial or lateral joint compartment. The introduction of the solution was administered slowly into the joint space or synovial space of the joint. This comprised the first injection of the method for prolonged treating chronic articular joint pain. A second injection was prepared and administered similarly to the first injection to the joint approximately 30 days later.

There have been described novel methods and compositions for prolonged alleviation of articular joint pain. It should be understood that the specific formulations and methods described herein are exemplary and should not be construed to limit the invention, which will be described in the claims below. Further, it is evident that those skilled in the art may now make numerous uses and modifications of the specific embodiments described without departing from the inventive concepts. For example, preparation of the treatment solution described herein, can also be prepared by any one of the other commonly known methods. Consequently, the invention is to be construed as embracing each and every novel feature and novel combination of features present in and/or possessed by the compositions and methods described and by their equivalents.

What is claimed:

1. A method for alleviating articular pain in a joint of a patient, comprising:
preparing a treatment solution containing Procaine and at least two corticosteroids; and
administering said treatment solution through an area of skin into a joint cavity of said joint.

2. The method of alleviating articular pain in a joint of claim 1 further comprising:
disinfecting said area of skin of said patient prior to said administering of said treatment solution.

3. The method of alleviating articular pain in a joint of claim 1 wherein said at least two corticosteroid is selected from the group consisting of joint compatible corticosteroids, connective tissue compatible corticosteroids, and synovia compatible corticosteroids.

4. The method of alleviating articular pain in a joint of claim 1 wherein said at least two corticosteroid is Betamethasone and Depomedrol.

5. The method of alleviating articular pain in a joint of claim 1 wherein said at least one anesthetic is present in said treatment solution in the range of from about 10 to about 70 percent by volume, based on the total volume of said treatment solution.

6. The method of alleviating articular pain in a joint of claim 1 wherein said at least one anesthetic is present in said treatment solution in the range of from about 30 to about 50 percent by volume, based on the total volume of said treatment solution.

7. The method of alleviating articular pain in a joint of claim 1 wherein said at least one anesthetic is 40 percent by volume in said treatment solution, based on the total volume of said treatment solution.

8. The method of alleviating articular pain in a joint of claim 1 wherein said at least two corticosteroid is each present in said treatment solution in the range of from about 10 to about 50 percent by volume, based on the total volume of said treatment solution.

9. The method of alleviating articular pain in a joint of claim 1 wherein said at least two corticosteroid is each present in said treatment solution in the range of from about 20 to about 40 percent by volume, based on the total volume of said treatment solution.

10. The method of alleviating articular pain in a joint of claim 1 wherein said at least two corticosteroid is each present in said treatment solution at 30 percent by volume, based on the total volume of said treatment solution.

11. The method of alleviating articular pain in a joint of claim 1 wherein said treatment solution is administered in the range of from about 0.1 cc to about 2.5 cc. in volume.

12. The method of alleviating articular pain in a joint of claim 1 wherein said treatment solution is 1.0 cc. in volume.

13. The method of alleviating articular pain in a joint of claim 1 wherein said administering comprises:
injecting said treatment solution into said joint of said patient.

14. The method of alleviating articular pain in a joint of claim 13 wherein said injecting is performed by a needle and syringe.

15. The method of alleviating articular pain in a joint of claim 14 wherein said needle is between ¾ and 1½ inches in length.

16. The method of alleviating articular pain in a joint of claim 14 wherein said needle is between 22 and 25 gauge in diameter.

17. The method of alleviating articular pain in a joint of claim 1 wherein said joint is selected from the group consisting of scapular humeral (shoulder) joint, elbow joint, carpal joint, stifle (knee) joint, coxofemoral (hip) joint, temporomandibular joint, and hock (ankle) joint.

18. The method of alleviating articular pain in a joint of claim 1 wherein said administering further comprises administering a first treatment solution into said joint cavity of said joint and then administering a second treatment solution into said joint cavity of said joint thirty days later.

19. The method of alleviating articular pain in a joint of claim 17 wherein further comprising:
flexing said scapular humeral (shoulder) joint and a bicipital tendon;
directing said administering of said treatment solution towards said joint space from an anterior lateral direction; and
administering said treatment solution slowly into said joint space.

20. The method of alleviating articular pain in a joint of claim 17 wherein further comprising:
flexing said elbow joint to approximately ninety degrees;
directing said administering of said treatment solution towards said joint space from at least one of a posterior lateral direction and a lateral direction; and
administering said treatment solution slowly into said joint space.

21. The method of alleviating articular pain in a joint of claim 17 wherein further comprising:
flexing said carpal (wrist) joint to provide an anterior approach to said carpal joint;
directing said administering of said treatment solution towards said joint space from an anterior direction to at least one of a medial and a lateral joint space; and
administering said treatment solution slowly into said joint space.

22. The method of alleviating articular pain in a joint of claim 17 wherein further comprising:
positioning said stifle (knee) joint to facilitate at least one of an anterior lateral and medial approach to said stifle joint;
directing said administering of said treatment solution towards said joint space from an anterior direction; and
administering said treatment solution slowly into said joint space.

23. The method of alleviating articular pain in a joint of claim 17 wherein further comprising:
positioning said coxofemoral (hip) joint to provide an anterior edge of a greater trochanter to be used as a guide for said administering of said treatment solution to said coxofemoral joint;
directing said administering of said treatment solution towards said joint space from an anterior to said greater trochanter;
guiding said administering of said treatment solution using a neck of a femoral head to move into said joint space; and
administering said treatment solution slowly into said joint space.

24. The method of alleviating articular pain in a joint of claim 17 wherein further comprising:
positioning said hock (ankle) joint so that said joint space of at least one of a tibial and tarsal joint to facilitate a posterior approach to said hock joint;
directing said administering of said treatment solution towards said joint space from an posterior direction to at least one of a medial and a lateral joint space; and
administering said treatment solution slowly into said joint space.

25. A method for alleviating articular pain in a joint of a patient, comprising:
preparing a treatment solution containing Procaine, Betamethasone, and Depomedrol; and
injecting said treatment solution through an area of skin into a joint cavity of said joint.

26. The method for alleviating articular pain in a joint of claim 25 further comprising:
disinfecting said area of skin of said patient prior to said administering of said treatment solution.

27. The method for alleviating articular pain in a joint of a patient of claim 25 wherein said Procaine is present in said treatment solution at 40 percent by volume in said treatment solution, based on the total volume of said treatment solution.

28. The method for alleviating articular pain in a joint of a patient of claim 25 wherein said Betamethasone is present in said treatment solution at 30 percent by volume in said treatment solution, based on the total volume of said treatment solution.

29. The method for alleviating articular pain in a joint of a patient of claim 25 wherein said Depomedrol is present in said treatment solution at 30 percent by volume in said treatment solution, based on the total volume of said treatment solution.

30. A pharmaceutical composition comprising:
Procaine, Betamethasone, and Depomedrol.

31. The pharmaceutical composition of claim 30 wherein said Procaine is present in said pharmaceutical composition in the range of from about 10 to about 70 percent by volume, said Betamethasone is present in said pharmaceutical composition in the range of from about 10 to about 50 percent by volume, and said Depomedrol is present in said pharmaceutical composition in the range of from about 10 to about 50 percent by volume, based on the total volume of said pharmaceutical composition.

32. The pharmaceutical composition of claim 30 wherein said Procaine is present in said pharmaceutical composition at 40 percent by volume, said Betamethasone is present in said pharmaceutical composition at 30 percent, and said Depomedrol is present in said pharmaceutical composition at 30 percent.

33. An articular joint pain prolonged alleviation kit, comprising:
a joint cavity delivery device;
in separate containers, Procaine; and at least two corticosteroids selected from the group consisting of joint compatible corticosteroids, connective tissue compatible corticosteroids, and synovia compatible corticosteroids.

34. The articular joint pain prolonged alleviation kit of claim 33 wherein said joint cavity delivery device is a syringe and needle.

35. The articular joint pain prolonged alleviation kit of claim 33 wherein said at least two corticosteroids are Betamethasone and Depomedrol.
